(19) United States
(12) Patent Application Publication
(10) Pub. No.: US 2025/0099463 A1
(43) Pub. Date: Mar. 27, 2025

(54) Sustained Release Pharmaceutical Composition of Upadacitinib

(71) Applicant: Renata Pharmaceuticals (Ireland) Limited, Dublin (IE)

(72) Inventors: Syed S. Kaiser Kabir, Dhaka (BD); Syed Omar Kabir, Dhaka (BD); Ganesh Vinayak Gat, Dhaka (BD); Rambabu Boorugu, Dhaka (BD); A.H.M. Masbahur Rahman, Dhaka (BD)

(21) Appl. No.: 18/729,541

(22) PCT Filed: Oct. 26, 2022
(86) PCT No.: PCT/EP2022/079990

§ 371 Date: Jul. 17, 2024

Related U.S. Application Data
(60) Provisional application No. 63/266,947, filed on Jan. 20, 2022.

(51) Int. Cl. A61K 31/4985; A61K 9/20

(52) U.S. Cl. CPC A61K 31/4985; A61K 9/2013

(57) ABSTRACT

Disclosed herein is a sustained release pharmaceutical composition of upadacitinib and process for preparing the same, wherein said pharmaceutical composition comprising therapeutically effective amount of upadacitinib and pharmaceutically acceptable adjuvants, and wherein the composition is free of polymer.

### CROSS REFERENCE TO RELATED APPLICATIONS

This application claims benefit of U.S. Provisional Application No. 63/266,947, filed Jan. 20, 2022, which application is incorporated herein by reference in its entirety.

### FIELD OF THE INVENTION

The present invention relates to a field of sustained release pharmaceutical compositions in general, and in particular to a sustained release pharmaceutical composition of upadacitinib and a process for preparing the same.

### BACKGROUND

Rheumatoid arthritis is an autoimmune disease that causes chronic inflammation of joints and other parts of the body and can cause permanent joint damage and deformities. If the disease is left untreated, it can lead to substantial disability and pain due to loss of joint function, ultimately leading to shortened life expectancy. JAK1 is a target for immune-inflammatory diseases, and its inhibitors are beneficial for the treatment of rheumatoid arthritis.

Upadacitinib is a second-generation oral JAK1 inhibitor developed by AbbVie. It has a high selectivity for JAK1 inhibition. The chemical name of the drug is: (3S, 4R)-3-ethyl-4-(3H-imidazo [1,2-a] pyrrolo [2,3-e] pyrazine-8-yl)-N-(2,2,2-trifluoroethyl) pyrrolidine-1-carboxamide, its structural formula is as follows:

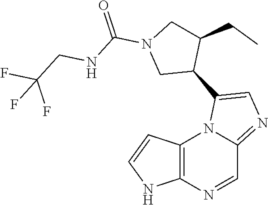 [SMILES: CC[C@@H]1CN(C(=O)NCC(F)(F)F)C[C@@H]1c1cnc2cnc3[nH]ccc3n12]

It is important that pharmaceutical products be effective and safe. Even if a pharmaceutical product is effective and safe immediately after production, if the drug is easily decomposed or denatured during distribution, it is not effective and safe as a pharmaceutical product. Therefore, the stability of the drug is extremely important for pharmaceutical products

In addition, to maintain effectiveness and safety of a pharmaceutical product, not only the effectiveness and safety of the active ingredient but also the properties of the pharmaceutical preparation, such as drug dissolution property in the body and the like, are extremely important. For example, when dissolution of the drug from the pharmaceutical preparation is too slow, the blood concentration of the drug does not reach an effective level, and the expected efficacy may not be sufficiently exhibited. On the other hand, when dissolution of the drug from the pharmaceutical preparation is too fast, the blood concentration of the drug increases rapidly, and the risk of side effects increases.

In other words, a pharmaceutical product is required to ensure stability and constant dissolution of drug, in addition to the effectiveness and safety.

Attempts have been made to provide sustained release formulations of anti-Alzheimer's agents. U.S. Pat. No. 9,963,459 discloses a sustained-release hydrophilic polymer matrix system of upadacitinib comprising a basic drug or a salt thereof, polymer and pH modifying agent. It further discloses that, the hydrophilic polymer, in contact with water, forms a gel layer that provides an environment suitable for upadacitinib and the pH modifier to dissolve. As present invention polymers forms gel and created multiple challenges regarding manufacturing process and analysis.

After rigorous experimentation it was surprisingly found that compositions substantially free of sustained release polymer comprising Upadacitinib or its salt thereof, and at least lipophilic release retardant, provide the desired sustained release properties and stability profile. The said composition is cost effective and process of obtaining it is less complex. Moreover, present invention is non-swelling solid oral composition with substantially free of sustained release polymer, thus does not form gel and hence easy to handle during manufacturing process and analysis.

### SUMMARY

In accordance with the principal aspect of the present invention, there is provided oral sustained release pharmaceutical composition comprising upadacitinib that exhibits sustained release of the drug.

In accordance with another aspect of the invention, there is provided a process for preparing oral sustained release pharmaceutical composition comprising upadacitinib that exhibits sustained release of the drug.

In accordance with one other aspect of the present invention, there is provided oral sustained release pharmaceutical composition comprising therapeutically effective amount of upadacitinib and pharmaceutically acceptable adjuvants, wherein the composition is substantially free of hydrophilic sustained release polymer.

In accordance with one other aspect of the present invention, there is provided oral sustained release pharmaceutical composition comprising therapeutically effective amount of upadacitinib and pharmaceutically acceptable adjuvants, wherein said composition contains lipophilic release retardants.

In accordance with yet another aspect of the present invention, there is provided oral sustained release pharmaceutical composition comprising a compressed core having therapeutically effective amount of fine upadacitinib and one or more pharmaceutically acceptable adjuvants, wherein said core is necessarily free of sustained release hydrophilic polymer and contains pH modifier.

In accordance with yet another aspect of the present invention, there is provided oral sustained release pharmaceutical composition of upadacitinib for oral administration, wherein said composition contains at least about 80% of un-degraded upadacitinib after storage for three months at 40° C.±20 C. and 75%±5% RH (relative humidity) in specialized packs.

In accordance with yet another aspect of the present invention, there is provided a sustained release pharmaceutical composition of upadacitinib for oral administration wherein the composition is manufactured in the controlled processing conditions.

In accordance with yet another aspect of the present invention, there is provided oral sustained release pharmaceutical composition of upadacitinib for oral administration, wherein the composition is prepared under controlled processing conditions comprising the steps of: (a) preparing a core, (b) optionally, forming a seal coating layer on the core and (c) optionally, forming a film coating on the seal coated core, and (d) packing final product in specialized packs.

In accordance with yet another aspect of the present invention, there is provided a composition is prepared under controlled processing conditions comprising the steps of: (a) preparing the core comprising (i) sifting upadacitinib and lipophilic release retardant(s); (ii) sifting optionally other pharmaceutical adjuvant(s), (iii) preparing granules by roller compaction (iv) lubricating the dried granulates with lubricant, (viii) compressing the lubricated granulates into a compressed core, (b) optionally forming the seal coating layer on the core, (c) optionally forming the non-functional film coating on the seal coated core and (d) packing the resultant product in specialized packs.

In accordance with yet another aspect of the present invention, there is provided a sustained release pharmaceutical composition of upadacitinib for oral administration, wherein the composition is formulated in various oral delivery devices, preferably tablet, capsule, granules, beads, or sachet.

The details of one or more embodiments of the inventions are set forth in the description below. Others features, objects and advantages of the inventions will be apparent from the description and claims.

### BRIEF DESCRIPTION OF DRAWINGS

FIG. 1 depicts swelling and gelling characteristic of Rinvoq™ tablet and tablet of Example 3.

FIG. 2 is a comparison of the dissolution profile at pH 6.8 of the extended release tablets from Examples 6-9.

### DETAILED DESCRIPTION

Before the present formulations and methods are described, it is to be understood that this invention is not limited to particular compounds, formulas or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a compound” includes a plurality of such compounds and reference to “the step” includes reference to one or more step and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In accordance with the principal aspect of the present invention, there is provided oral sustained release pharmaceutical composition comprising upadacitinib that exhibits sustained release of the drug.

In accordance with another aspect of the invention, there is provided a process for preparing said oral sustained release pharmaceutical composition comprising upadacitinib that exhibits sustained release of the drug.

In accordance with one other aspect of the present invention, there is provided oral sustained release pharmaceutical composition comprising therapeutically effective amount of upadacitinib and pharmaceutically acceptable adjuvants, wherein the composition is substantially free of hydrophilic sustained release polymer.

In accordance with one other aspect of the present invention, there is provided oral sustained release pharmaceutical composition comprising therapeutically effective amount of upadacitinib and pharmaceutically acceptable adjuvants, wherein said composition contains lipophilic release retardants.

In accordance with yet another aspect of the present invention, there is provided oral sustained release pharmaceutical composition comprising a compressed core having therapeutically effective amount of fine upadacitinib and one or more pharmaceutically acceptable adjuvants, wherein said core is necessarily free of sustained release hydrophilic polymer and contains pH modifier.

In accordance with yet another aspect of the present invention, there is provided oral sustained release pharmaceutical composition of upadacitinib for oral administration, wherein said composition contains at least about 80% of un-degraded upadacitinib after storage for three months at 40° C.±20 C. and 75%±5% RH (relative humidity) in specialized packs.

In accordance with yet another aspect of the present invention, there is provided sustained release pharmaceutical composition of upadacitinib for oral administration wherein the composition is manufactured in the controlled processing conditions.

In accordance with yet another aspect of the present invention, there is provided oral sustained release pharmaceutical composition of upadacitinib for oral administration, wherein the composition is prepared under controlled processing conditions comprising the steps of: (a) preparing a core, (b) optionally, forming a seal coating layer on the core and (c) optionally, forming a film coating on the seal coated core, and (d) packing final product in specialized packs.

In accordance with yet another aspect of the present invention, there is provided a sustained release pharmaceutical composition of upadacitinib for oral administration, wherein the composition is prepared under controlled processing conditions comprising the steps of: (a) preparing the core comprising (i) sifting upadacitinib and lipophilic release retardant(s); (ii) sifting optionally other pharmaceutical adjuvant(s), (iii) preparing granules by roller compaction (iv) lubricating the dried granulates with lubricant, (viii) compressing the lubricated granulates into a compressed core, (b) optionally forming the seal coating layer on the core, (c) optionally forming the non-functional film coating on the seal coated core and (d) packing the resultant product in specialized packs.

In accordance with yet another aspect of the present invention, there is provided composition is formulated in various oral delivery devices, preferably tablet, capsule, granules, beads, or sachet.

The details of one or more embodiments of the inventions are set forth in the description below. Others features, objects and advantages of the inventions will be apparent from the description and claims.

The term “upadacitinib” or a pharmaceutically acceptable salt thereof refers to upadacitinib free base or any pharmaceutically acceptable salt of upadacitinib, the controlled release pharmaceutical composition of the present invention may include upadacitinib in amounts ranging from about 10% w/w to about 50% w/w of the composition, 1% w/w to about 50% w/w of the composition and 2% w/w to about 10% w/w of the composition.

The controlled release pharmaceutical composition of the present invention may optionally include one or more coating layer(s). Suitable coloring agents include any FDA approved colors for oral use.

The controlled release pharmaceutical composition of the present invention may include one or more pharmaceutically acceptable excipients such as diluents, binders, disintegrants, lubricants, glidants, plasticizers, stabilizers, and coloring agents in coating layer.

Lipophilic release retardants are non-swelling and non-gelling release retardant. Some of the lipophilic release retardant are also found as monomer, dimer or trimer. Glyceyl behenate is mixture of glycerol esters. As per PhEur 6.0, glyceryl dibehenate as a mixture of diacyglycerols, mainly dibehenoylglycerol, together with variable quantities of mono- and triacylglycerols. As per USP32-NF27, glyceryl behenate as a mixture of glycerides of fatty acids, mainly behenic acids and content of 1-monoglycerides should be 12.0-18.0%. Unlimited examples of lipophilic release retardants in accordance with the present invention include, but not limited to, hydrogenated oils such as, hydrogenated vegetable oil, cottonseed oil, castor oil, canola oil, palm oil, palm kernel oil and soybean oil, cetostearyl alcohol, cetyl alcohol, glyceryl behenate derivatives (such as Compritol® ATO888, Compritol® HD ATO5), glyceryl mono oleate, glyceryl mono stearates, glyceryl palmito stearates (such as Precirol® ATO5, lecithin, mono-di- and triglycerides with polyethylene glycol (PEG) esters of fatty acid (such as Gelucire® 54/02, 50/13, 43/01), medium chain triglycerides, carnauba wax, microcrystalline wax, beeswax, any combination thereof and the like. Other forms of sustained release agents are also contemplated. In particular, hydrogenated castor oil (commercially available under the brand name Cutina®HR from Cognis, North America) is found to be useful.

According to the present invention, the effective amount of lipophilic release retardant required to achieve sustained release of upadacitinib may vary between 5% to 55% or 25% to 55%, or 30% to 50% and 35% to 45% of the uncoated tablet weight. In general, any amount that will effectively demonstrate a sustained release profile of the upadacitinib can be used.

Unless otherwise stated all concentrations mentioned herein are based on total weight of the composition.

The ratio of upadacitinib to lipophilic release retardant material ranges from about 0.5 to 2 or 0.05 to 2. The concentration of lipophilic material used is reasonably equal, allowing formation of very hard tablets, which can withstand various rigors. It is believed, without wishing to be bound by any theory that, the use of lipophilic release retardant material having melting point higher than human body temperature contributes to the stability of the dosage form.

Suitable diluents that may be used in the composition of the present invention include one or more of calcium carbonate, calcium phosphate dibasic, calcium phosphate-tribasic, calcium sulfate, microcrystalline cellulose, silicified microcrystalline cellulose, cellulose powdered, dextrates, dextrins, dextrose excipients, fructose, kaolin, lactitol, lactose, mannitol, sorbitol, starch, starch pregelatinized, sucrose, sugar compressible, sugar confectioners and the like and mixtures thereof. The diluents may be present in the composition of the present invention in an amount ranging from about 5% w/w to about 75% w/w of the composition.

The binders that may be used in the composition of the present invention include one or more of methyl cellulose, hydroxypropyl cellulose, hydroxypropyl methyleellulose, polyvinylpyrrolidone, gelatin, gum arabic, ethyl cellulose, polyvinyl alcohol, pullulan, pregelatinized starch, agar, tragacanth, sodium alginate, propylene glycol and the like and mixtures thereof. The binders may be present in the composition of the present invention in an amount ranging from about 1% w/w to about 10% w/w of the composition.

Suitable disintegrants that may be used in the composition of the present invention include one or more of starch, croscarmellose sodium, crospovidone, sodium starch glycolate, potassium polacrillm and the like and mixtures thereof. The disintegrants may be present in the composition of the present invention in an amount ranging from about 1% w/w to about 25% w/w of the composition.

The lubricants and glidants that may be used in the composition of the present invention include one or more of colloidal anhydrous silica, stearic acid, magnesium stearate, calcium stearate, talc, hydrogenated castor oil, sucrose esters of fatty acid, microcrystalline wax, yellow beeswax, white beeswax and the like and mixtures thereof. The lubricants and glidants may be present in the composition of the present invention in an amount ranging from about 0.05% w/w to about 5% w/w of the composition. Suitable plasticizers that may be used in the composition of the present invention include one or more of polyethylene glycol, triethyl citrate, triacetin, diethyl phthalate, dibutyl sebacetate, hydrogenated vegetable oil such as Lubritab, polyoxyethylene alkyl ethers such as Cremophor and the like and mixtures thereof. The plasticizers may be present in the composition of the present invention in an amount ranging from about 1% w/w to about 10% w/w of the composition.

Suitable stabilizers include one or more of antioxidants, buffers and the like may also be used in the composition of the present invention. The stabilizers may be present in the composition of the present invention in an amount ranging from about 0.05% w/w to about 10% w/w of the composition.

As the buffering agent to be used in the present invention, any buffering agent can be used as long as it can simultaneously achieve the stability of upadacitinib in a drug product and dissolution property thereof from the drug product, and is applicable to pharmaceutical products. Plural buffering agents may be used in combination. As the buffering agent to be used in the present invention, a buffering agent showing pH of about 2 to about 5, preferably about 3 to about 5, more preferably about 3 to about 4 is preferably used. For example, an acidic substance such as tartaric acid, citric acid, lactic acid, fumaric acid, malic acid, ascorbic acid, acetic acid, acidic amino acid (e.g., glutamic acid, aspartic acid) and the like, inorganic salts of these acidic substances (e.g., alkali metal salt, alkaline earth metal salt, ammonium salt, etc.), salts of these acidic substances with an organic base (e.g., basic amino acid such as lysine, arginine, etc., meglumine, etc.), and a hydrate thereof, a solvate thereof and the like are used.

Here, the pH of the buffering agent is measured under the following conditions. To be precise, it is a pH of a solution or suspension obtained by dissolving or suspending a buffering agent in water at a concentration of 1% w/v at 25° C.

As the buffering agent to be used in the present invention, an acidic substance and a basic substance are combined, and the obtained buffering agent may be adjusted such that the pH of a solution or suspension is about 2 to about 5, preferably about 3 to about 5, more preferably about 3 to about 4, when the combined buffering agent is dissolved or suspended in water at 25° C. at a concentration of 1% w/v. Examples of the acidic substance to be used in combination include, in addition to the acidic substances having a pH of about 2 to about 5 mentioned above and salts thereof, strong acids such as hydrochloric acid, sulfuric acid, phosphoric acid and like. Examples of the basic substance to be used in combination include inorganic bases (e.g., sodium hydroxide, potassium hydroxide, sodium carbonate, sodium hydrogencarbonate, magnesium carbonate, calcium carbonate, magnesium oxide, ammonia, synthetic hydrotalcite), organic bases (e.g., basic amino acid such as lysine, arginine, etc., meglumine, and the like) and the like.

Furthermore, examples of the buffering agent to be used in the present invention include those whose solutions have a buffering ability at said pH, such as sodium dihydrogen phosphate, monosodium fumarate and the like.

As the buffering agent to be used in the present invention, monosodium fumarate and fumaric acid and sodium hydroxide may be used in combination.

The solid pharmaceutical composition of the present invention contains a buffering agent at a proportion of 0.01-20 wt %, preferably 0.05-10 wt %, more preferably 0.1-5 wt %.

The following examples are provided to enable one of ordinary skill in the art to prepare dosage forms of the invention and should not be construed as limiting the scope of the invention.

### Example 1-4

TABLE NO 1

| | Example No. | | | |
|---|---|---|---|---|
| | Exam- | Exam- | Exam- | Exam- |
| | ple 1 | ple 2 | ple 3 | ple 4 |
| Upadacitinib | 15.00 | 15.00 | 15.00 | 15.00 |
| Glyceryl Dibehenate | 48.00 | 96.00 | 120.00 | 144.00 |
| (Compritol 888 ATO) | | | | |
| Microcrystalline Cellulose | 206.80 | 174.80 | 158.70 | 142.70 |
| PH 112 | | | | |
| Mannitol (Pearlitol SD | 103.40 | 87.40 | 79.50 | 71.50 |
| 200SD) | | | | |
| Tartaric Acid | 96.00 | 96.00 | 96.00 | 96.00 |
| Colloidal Silicon Dioxide | 2.40 | 2.40 | 2.40 | 2.40 |
| Magnesium Stearate | 3.60 | 3.60 | 3.60 | 3.60 |
| Colloidal Silicon Dioxide | 1.20 | 1.20 | 1.20 | 1.20 |
| Magnesium Stearate | 3.60 | 3.60 | 3.60 | 3.60 |
| Total | 480.00 | 480.00 | 480.00 | 480.00 |

Manufacturing Process for Example 1-4:
1. Upadacitinib and Microcrystalline cellulose PH 112 were mixed geometrically and mixture were sieved through 40 #ASTM.
2. Compritol 888 ATO, Colloidal Silicon Dioxide, Mannitol 200 SD and Tartaric Acid were sieved through 40 #ASTM and mixed with blend of step 1 using bin blender 10 minutes at 10 rpm.
3. Magnesium Stearate was sieved through 60 #ASTM and used for lubricating blend of step-2 in a bin blender for 3 minutes at 10 rpm.
4. Slug the lubricated materials of step 3 in a roller compactor with following parameters:
   a. Roller speed: Set: 4 RPM; Actual: 3.8-4.2
   b. Screw speed: Set: 60 RPM; Actual: 57-63
   c. Hydraulic Pressure: Set: 50 Bar; Actual: 49-51
   d. Roller gap: Actual: 1.2-2
5. The slugged material was milled using co-mill through 12 #ASTM followed by 20 #ASTM.
6. Colloidal Silicon Dioxide, Magnesium Stearate were sieved through 40 #ASTM.
7. The compacted granules and extragranular Colloidal Silicon Dioxide were blended in a bin blender for 10 minutes at 10 rpm.
8. Magnesium stearate was added to blend of step 7 and lubricated in a bin blender at 10 rpm for 3 minutes.
9. Tablet were produced using compression machine.
10. Compressed tablet were coated with PVA based coating material for 3% weight gain.

### Example 5

Swelling study were conducted using the dissolution Apparatus on Rinvoq™ tablet and Tablet of Example 3, without rotation speed, in 200 mL of pH 6.8 Phosphate Buffer at 37.0±0.5° C. At predetermined time intervals of 1, 2, 3, 4, 6, 8 hours, the swollen tablets were removed from the buffer media, immediately wiped with a paper towel to remove surface droplets, and weighed. The swelling index (SI) was calculated according to the following equation:

$$SI ( \% ) = \frac{Wt - Wo}{Wo} \times 100$$

where Wo is the initial weight of the dry tablet and Wt is the weight of the swollen tablet at time t.

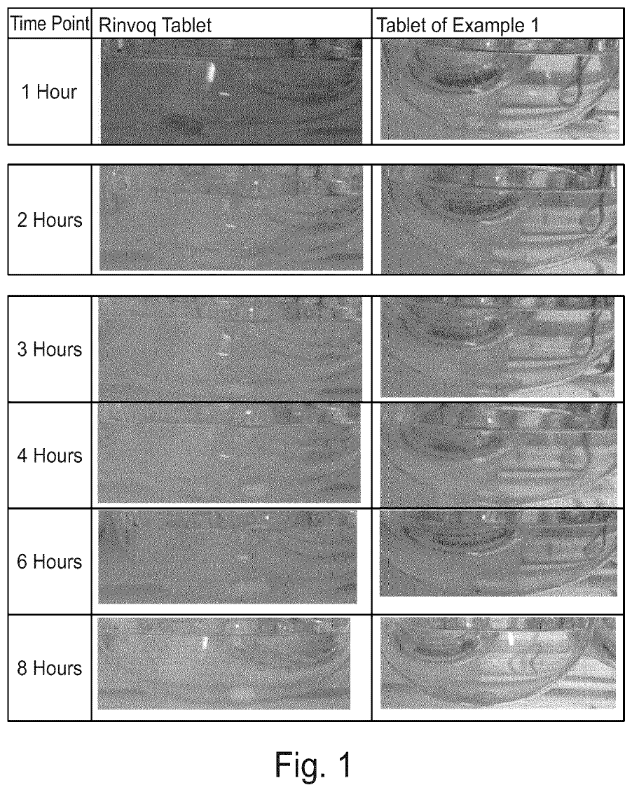

The Rinvoq™ tablet swells and gels but Tablet of example 3 does not swell and swelling index at different time point is given in Tablet no. 2 and pictures of same given in FIG. 1.

TABLE NO 2

| Time | Rinvoq Tablet | | Tablet of Example 3 | |
|---|---|---|---|---|
| (hrs) | Weight (g) | Swelling Index | Weight (g) | Swelling Index |
| 0 | 0.498 | 0.00 | 0.487 | 0.00 |
| 1 | 0.698 | 40.16 | 0.485 | −0.41 |
| 2 | 0.792 | 59.04 | 0.497 | 2.05 |
| 3 | 0.795 | 59.64 | 0.494 | 1.44 |
| 4 | 0.821 | 64.86 | 0.502 | 3.08 |
| 6 | 0.833 | 67.27 | 0.506 | 3.90 |
| 8 | 0.834 | 67.47 | 0.503 | 3.29 |

### Example 6-9

TABLE NO 3

| | | | | | |
|---|---|---|---|---|---|
| Sl. | Name of the | Example-6 | Example-7 | Example-8 | Example-9 |
| No | Material | Qty/tab | Qty/tab | Qty/tab | Qty/tab |

Intra-Granular Materials

| | | | | | |
|---|---|---|---|---|---|
| 1 | Upadacitinib | 45 | 45 | 45 | 45 |
| 2 | Glyceryl Dibehenate | 95 | 105 | 120 | 130 |
| | (Compritol 888 | | | | |
| | ATO) | | | | |
| 3 | Microcrystalline | 177.9 | 142.9 | 142.9 | 142.9 |
| | Cellulose PH 112 | | | | |
| 4 | Mannitol (Pearlitol | 60.3 | 60.3 | 60.3 | 60.3 |
| | 200 SD) | | | | |
| 5 | Tartaric Acid | 96 | 96 | 96 | 96 |
| 6 | Colloidal Silicon | 2.4 | 2.4 | 2.4 | 2.4 |
| | Dioxide | | | | |
| 7 | Magnesium Stearate | 3.6 | 3.6 | 3.6 | 3.6 |

Extra Granular Material

| | | | | | |
|---|---|---|---|---|---|
| 8 | Colloidal Silicon | 1.2 | 1.2 | 1.2 | 1.2 |
| | Dioxide | | | | |
| 9 | Magnesium Stearate | 3.6 | 3.6 | 3.6 | 3.6 |

| | | | | |
|---|---|---|---|---|
| Core Tablet weight | 485 | 460 | 475 | 485 |

Coating

| | | | | | |
|---|---|---|---|---|---|
| 10 | Opadry II Yellow | 14.55 | 13.8 | 14.25 | 14.55 |
| | Ph. Gr. | | | | |

| | | | | |
|---|---|---|---|---|
| Coated Tablet weight | 499.55 | 473.8 | 489.25 | 499.55 |

Manufacturing Process for Example 6-9, was followed, same as described above for Examples 1-4.

The effect of solid state form on the dissolution profile of the tablets was evaluated. In particular, the dissolution profile of the Example 6 to 9 tablets was evaluated at pH 6.8. The dissolution test was carried out using the following dissolution parameters and conditions:
Apparatus: USP Dissolution Apparatus 2 and fraction collector
Medium: 900 mL of 50 mM sodium phosphate buffer solution, pH 6.8.
Temperature: 37° C.±0.5° C.
RPM: 100 RPM=4%
Filter: 35 μm PE filter, or equivalent, for automatic sampling
Sampling Times: 1, 4, 12 and 24 Hours.
Sample Volume: 1.5 mL obtained automatically, without media replacement.

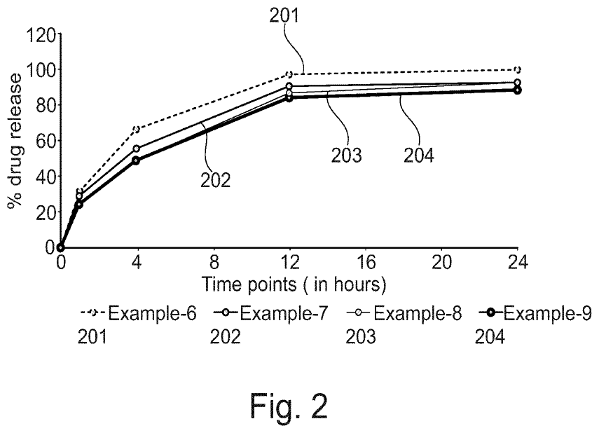

For the analysis of the sample, conventional liquid chromatography methods were utilized, wherein the % of the labelled amount of drug released (% Drug Released) was calculated. The results are set forth in FIG. 2.

We claim:

1. An extended release solid dosage form of a pharmaceutical composition comprising
a) upadacitinib or a pharmaceutically acceptable salt thereof;
b) lipophilic release retardant and
c) optionally one or more excipient.

2. The pharmaceutical composition according to claim 1, wherein the composition further contains 5% to 55% of lipophilic release retardant.

3. The pharmaceutical composition according to claim 1, wherein said lipophilic release retardant(s) are selected from one or more hydrogenated oils comprising from hydrogenated vegetable oil, cottonseed oil, castor oil, canola oil, palm oil, palm kernel oil and soybean oil, cetostearyl alcohol, cetyl alcohol, glyceryl behenate derivatives, glyceryl mono oleate, glyceryl mono stearates, glyceryl palmito stearates, lecithin, mono-di- and triglycerides with polyethylene glycol (PEG) esters of fatty acid, medium chain triglycerides, carnauba wax, microcrystalline wax, beeswax or any combination thereof.

4. The pharmaceutical composition according to claim 1, wherein the lipophilic release retardant is glyceryl behenate derivatives.

5. The extended release solid dosage form of claim 1, wherein one or more excipient is pH modifier.

6. The extended release solid dosage form of claim 1, wherein one or more excipient is an organic acid.

7. The extended release solid dosage form of claim 6, wherein the organic acid is present in an amount from about 10 w/w % to about 35 w/w %.

8. The extended release solid dosage form of claim 7, wherein the organic acid is tartaric acid.

9. The extended release solid dosage form of claim 6, wherein the extended release formulation provides for the release of upadacitinib or a pharmaceutically acceptable salt thereof upon entry into a use environment at a rate substantially independent of the pH of the use environment, wherein the use environment has a pH range from about 1.2 to about 6.8.

10. The extended release solid dosage form of claim 9, wherein the organic acid is tartaric acid.

11. An extended release solid dosage form comprising
a) upadacitnib freebase equivalent;
b) an acidic pH modifier; and
c) a lipophilic release retardant.

12. The pharmaceutical composition according to claim 1, wherein the composition further contains 5% to 55% of lipophilic release retardant.

13. The extended release solid dosage form of claim 11, wherein said lipophilic release retardant(s) are selected from one or more hydrogenated oils comprising from hydrogenated vegetable oil, cottonseed oil, castor oil, canola oil, palm oil, palm kernel oil and soybean oil, cetostearyl alcohol, cetyl alcohol, glyceryl behenate derivatives, glyceryl mono oleate, glyceryl mono stearates, glyceryl palmito stearates, lecithin, mono-di- and triglycerides with polyethylene glycol (PEG) esters of fatty acid, medium chain triglycerides, carnauba wax, microcrystalline wax, beeswax or any combination thereof.

14. The extended release solid dosage form of claim 12, wherein the lipophilic release retardant is glyceryl behenate derivatives.

15. The extended release solid dosage form of claim 13, wherein the organic acid is tartaric acid.

16. The extended release solid dosage form of claim 12, wherein the extended release formulation provides for the release of upadacitinib upon entry into a use environment at a rate substantially independent of the pH of the use environment, wherein the use environment has a pH range from about 1.2 to about 6.8.

17. The extended release solid dosage form of claim 15, wherein the organic acid is present in an amount from about 10 w/w % to about 35 w/w %.

18. The extended release solid dosage form of claim 16, wherein the organic acid is tartaric acid.

19. The extended release solid dosage form of claim 6, wherein the extended release formulation provides for the release of upadacitinib or a pharmaceutically acceptable salt thereof upon entry into a use environment at a rate substantially independent of the pH of the use environment, wherein the use environment has a pH range from about 1.2 to about 6.8.

20. The extended release solid dosage form of claim 15, wherein the extended release solid dosage form is tablet.
